(19) United States
(12) Patent Application Publication
(10) Pub. No.: US 2006/0107470 A1
(43) Pub. Date: May 25, 2006

(54) Composition for the oxidation dyeing of keratin fibers, comprising a 3,5-diaminopyridine derivative and a cationic or amphoteric polymer

(75) Inventor: Marie-Pascale Audouset, Asnieres (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal S.A.

(21) Appl. No.: 11/325,516

(22) Filed: Jan. 5, 2006

Related U.S. Application Data
(63) Continuation of application No. 10/257,418, filed on Oct. 11, 2002.

(30) Foreign Application Priority Data
Apr. 12, 2000 (FR) FR 00/04720

(51) Int. Cl. A61K 8/00

(52) U.S. Cl. 8/405

(57) ABSTRACT

The invention concerns an oxidation dyeing composition for keratinous fibres, in particular human keratinous dyeing such as hair comprising, in a medium suitable for dyeing, at least a coupling agent selected among 3,5-diamino-pyridine derivatives and their addition salts with an acid, at least an oxidation base, and at least a particular cationic or amphoteric polymer. The invention also concerns dyeing methods and devices using said composition.

The present invention relates to a composition for the oxidation dyeing of keratin fibers, and in particular of human keratin fibers such as the hair, comprising, in a medium that is suitable for dyeing, at least one coupler chosen from 3,5-diaminopyridine derivatives and the addition salts thereof with an acid, at least one oxidation dye precursor and at least one particular cationic or amphoteric polymer defined below.

It is known practice to dye keratin fibers, and in particular human hair, with dye compositions containing oxidation dye precursors, generally known as “oxidation bases”, in particular ortho- or para-phenylenediamines, ortho- or para-aminophenols and heterocyclic bases.

Oxidation dye precursors are compounds which are initially uncolored or only weakly colored and which develop their dyeing power on the hair in the presence of oxidizing agents leading to the formation of colored compounds. The formation of these colored compounds results either from an oxidative condensation of the “oxidation bases” with themselves or from an oxidative condensation of the “oxidation bases” with coloration modifiers, or “couplers”, which are generally present in the dye compositions used in oxidation dyeing and are represented more particularly by meta-phenylenediamines, meta-aminophenols and meta-diphenols, and certain heterocyclic compounds.

The variety of molecules used, which consist on the one hand of the “oxidation bases” and on the other hand of the “couplers”, allows a very wide range of colors to be obtained.

The “permanent” coloration obtained by means of these oxidation dyes must moreover satisfy a certain number of requirements. Thus, it must allow shades to be obtained with the desired intensity and must have good resistance to external agents (light, bad weather, washing, permanent waving, perspiration and rubbing).

The dyes must also allow gray hair to be covered, and finally they must be as unselective as possible, i.e. they must produce the smallest possible color differences along a same length of keratin fiber, which may in fact be differently sensitized (i.e. damaged) between its end and its root.

Thus, oxidation dye compositions containing certain 3,5-diaminopyridine derivatives as coupler, in combination with oxidation bases conventionally used in oxidation dyeing, have already been proposed, especially in patents U.S. Pat. No. 4,473,375 or DE-3 132 885.

However, the colorations obtained using these compositions are not always sufficiently strong, chromatic or resistant to the various attacking factors to which the hair may be subjected.

The Applicant has now discovered that it is possible to obtain, entirely surprisingly and unexpectedly, novel oxidation dyes capable of giving chromatic, strong, esthetic, unselective colorations in varied shades, that show good resistance to the various attacking factors to which the fibers may be subjected, by combining at least one coupler chosen from the 3,5-diaminopyridines of formula (I) defined below and the addition salts thereof with an acid, at least one oxidation dye precursor and at least one particular cationic or amphoteric polymer defined below.

This discovery forms the basis of the present invention.

A first subject of the invention is thus a composition for the oxidation dyeing of keratin fibers, and in particular of human keratin fibers such as the hair, comprising, in a medium that is suitable for dyeing,
(a) as coupler, at least one 3,5-diaminopyridine derivative of formula (I) below: 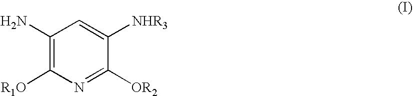
in which

R₁ and R₂, which may be identical or different, represent a C₁-C₄ alkyl, C₁-C₄ monohydroxyalkyl or C₂-C₄ polyhydroxyalkyl radical,

R₃ represents a hydrogen atom or a C₂-C₄ alkyl, C₂-C₄ monohydroxyalkyl or C₂-C₄ polyhydroxyalkyl radical, or one of the addition salts thereof with an acid; and

(b) at least one oxidation dye precursor, characterized in that it also comprises at least one cationic or amphoteric polymer chosen from the group formed by:

(i) methacryloyloxyethyltrimethylammonium halide homopolymers and copolymers;

(ii) polyquaternary ammoniums comprising repeating units of formula (I) or (II) below:

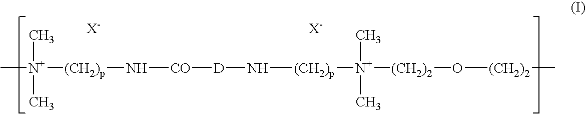

in which p denotes an integer ranging from 1 to 6 approximately, D may be zero or may represent a group —(CH₂)ᵣ—CO— in which r denotes a number equal to 4 or to 7, and X⁻ is an anion; 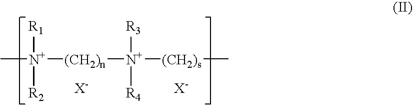
in which R₁, R₂, R₃ and R₄, which may be identical or different, denote an alkyl or hydroxyalkyl radical containing from 1 to 4 carbon atoms approximately, n and s are integers ranging from 2 to 20 and X⁻ is an anion;
(iii) vinylpyrrolidone copolymers containing cationic units;
(iv) amino silicones;
(v) dimethyldiallylammonium halide homopolymers and copolymers of dimethyldiallylammonium halide and of acrylamide;
(vi) copolymers of dimethyldiallylammonium halide and of acrylic acid;
(vii) copolymers of (meth)acrylic acid, or of maleic acid, or of α-chloroacrylic acid and of dialkylaminoalkyl (meth)acrylate or of dialkylaminoalkyl(meth)acrylamide;
(viii) the copolymer of sodium acrylate and of acrylamidopropyltrimethylammonium chloride;
(ix) octylacrylamide/acrylates/butylaminoethyl methacrylate copolymer;
(x) polymers comprising zwitterionic units of formula (III) below: 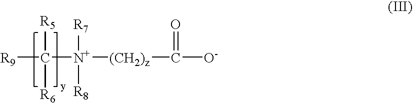
in which R₉ denotes an acrylate, methacrylate, acrylamide or methacrylamide group, y and z represent an integer from 1 to 3, R₅ and R₆ represent a hydrogen atom, methyl, ethyl or propyl, R₇ and R₈ represent a hydrogen atom or an alkyl radical such that the sum of the carbon atoms in R₇ and R₈ does not exceed 10;
(xi) the polymers derived from chitosan described especially in French patent 2 137 684 or U.S. Pat. No. 3,879,376, comprising monomer units corresponding to formulae (IV), (V) and (VI) below combined in their chain: 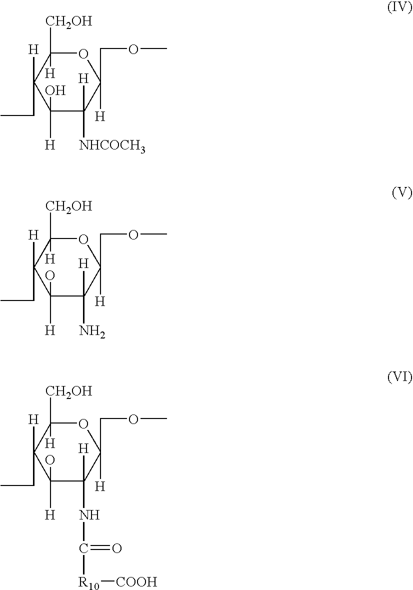
the unit (IV) being present in proportions of between 0 and 30%, the unit (V) in proportions of between 5% and 50% and the unit (VI) in proportions of between 30% and 90%, it being understood that, in this unit (VI), R₁₀ represents a radical of formula: 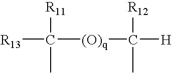
in which q denotes zero or 1;
if q=0, R₁₁, R₁₂ and R₁₃, which may be identical or different, each represent a hydrogen atom, a methyl, hydroxyl, acetoxy or amino residue, a monoalkylamine residue or a dialkylamine residue optionally interrupted with one or more nitrogen atoms and/or optionally substituted with one or more amine, hydroxyl, carboxyl, alkylthio or sulfonic groups, an alkylthio residue in which the alkyl group bears an amino residue, at least one of the radicals R₁₁, R₁₂ and R₁₃ being in this case a hydrogen atom; or if q=1, R₁₁, R₁₂ and R₁₃ each represent a hydrogen atom, and also the salts formed by these compounds with bases or acids.

Polymers of this type that are more particularly preferred comprise from 0 to 20% by weight of units (IV), from 40% to 50% by weight of units (V) and from 40% to 50% by weight of units (VI) in which R₁₀ denotes a —CH₂—CH₂— radical;
(xii) polymers corresponding to general formula (VII): 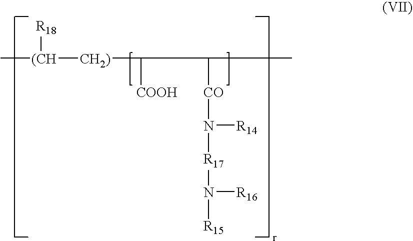
in which R₁₈ represents a hydrogen atom or a CH₃O, CH₃CH₂O or phenyl radical, R₁₄ denotes hydrogen or a (C₁-C₆)alkyl radical, R₁₅ denotes hydrogen or a (C₁-C₆)alkyl radical, R₁₆ denotes a (C₁-C₆)alkyl radical, or a radical corresponding to the formula: —R₁₇—N(R₁₅)₂, R₁₇, representing a —CH₂—CH₂—, —C₂—CH₂—CH₂— or —CH₂—CH(CH₃—)— group, and R₁₅ having the meanings given above;
(xiii) copolymers of (C₁-C₅)alkyl vinyl ether/maleic anhydride partially modified by semiamidation with an N,N-dialkylaminoalkylamine or by semiesterification with an N,N-dialkanolamine.

The dye composition in accordance with the invention thus defined gives, after mixing with an oxidizing composition, chromatic, strong, esthetic colorations in varied shades, showing low selectivity and excellent properties of resistance not only to atmospheric agents such as light and bad weather, but also to perspiration and the various treatments to which the hair may be subjected.

Another subject of the invention relates to a ready-to-use composition for the oxidation dyeing of keratin fibers, which comprises, in a medium that is suitable for dyeing, at least one 3,5-diaminopyridine of formula (I), at least one oxidation dye precursor, at least one cationic or amphoteric polymer defined above and at least one oxidizing agent.

For the purposes of the present invention, the expression “ready-to-use composition” means any composition intended to be applied immediately to keratin fibers.

The invention is also directed toward a process for the oxidation dyeing of keratin fibers, and in particular of human keratin fibers such as the hair, which consists in applying to the fibers a dye composition containing, in a medium that is suitable for dyeing, at least one 3,5-diaminopyridine of formula (I) and at least one oxidation dye precursor, the color being developed at alkaline, neutral or acidic pH with the aid of an oxidizing composition that is mixed with the dye composition just at the time of use, or that is applied sequentially without intermediate rinsing, at least one cationic or amphoteric polymer defined according to the invention being present in the dye composition and/or oxidizing composition.

A subject of the invention is also multi-compartment dyeing devices or “kits” for the oxidation dyeing of keratin fibers, and in particular of human keratin fibers such as the hair.

Such devices comprise a first compartment containing at least one 3,5-diaminopyridine of formula (I) and at least one oxidation dye precursor and a second compartment containing an oxidizing agent, at least one cationic or amphoteric polymer defined according to the invention being present in the first compartment and/or in the second compartment.

Another multi-compartment dyeing device comprises at least one compartment containing at least one 3,5-diaminopyridine of formula (I) and at least one oxidation dye precursor, at least one compartment containing at least one cationic or amphoteric polymer defined according to the invention, and at least one other compartment containing at least one oxidizing agent.

However, other characteristics, aspects, objects and advantages of the invention will emerge even more clearly on reading the description and the examples that follow.

Among the 3,5-diaminopyridine derivatives of formula (I) in accordance with the invention, mention may be made of 2,6-dimethoxy-3,5-diaminopyridine, 2,6-diethoxy-3,5-diaminopyridine and 2,6-di(β-hydroxyethyloxy)-3,5-diaminopyridine, and the addition salts thereof with an acid.

According to the invention, the dye composition preferably contains 2,6-dimethoxy-3,5-diaminopyridine, or at least one of the addition salts thereof with an acid.

The 3,5-diaminopyridine derivative(s) of formula (I) that may be used in the dye composition in accordance with the invention preferably represent(s) from 0.0001% to 10% by weight approximately relative to the total weight of the dye composition, and even more preferably from 0.005% to 5% by weight approximately relative to this weight.

The dye composition in accordance with the invention contains at least one oxidation dye precursor or oxidation base.

The nature of these oxidation bases is not critical.

They may especially be chosen from para-phenylenediamines, double bases, para-aminophenols, ortho-aminophenols and heterocyclic oxidation bases, and the addition salts thereof with an acid.

Among the para-phenylenediamines that may be used as oxidation base in the dye compositions in accordance with the invention, mention may be made especially of the compounds of formula (II) below, and the addition salts thereof with an acid: 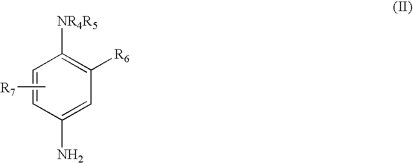
in which:

R₄ represents a hydrogen atom, a C₁-C₄ alkyl radical, a C₁-C₄ monohydroxyalkyl radical, a C₂-C₄ polyhydroxyalkyl radical, a (C₁-C₄)alkoxy(C₁-C₄)alkyl radical, a C₁-C₄ alkyl radical substituted with a nitrogenous group, a phenyl radical or a 4′-aminophenyl radical;

R₅ represents a hydrogen atom, a C₁-C₄ alkyl radical, a C₁-C₄ monohydroxyalkyl radical, a C₁-C₄ polyhydroxyalkyl radical, a (C₁-C₄)alkoxy(C₁-C₄)alkyl radical or a C₂-C₄ alkyl radical substituted with a nitrogenous group;

R₆ represents a hydrogen atom, a halogen atom such as a chlorine, bromine, iodine or fluorine atom, a C₁-C₄ alkyl radical, a C₁-C₄ monohydroxyalkyl radical, a C₁-C₄ hydroxyalkoxy radical, an acetylamino(C₁-C₄)alkoxy radical, a C₁-C₄ mesylaminoalkoxy radical or a carbamoylamino(C₁-C₄)alkoxy radical,

R₇ represents a hydrogen or halogen atom or a C₁-C₄ alkyl radical.

Among the nitrogenous groups of formula (II) above, mention may be made especially of amino, mono(C₁-C₄)alkylamino, di(C₁-C₄)alkylamino, tri(C₂-C₄)alkylamino, monohydroxy(C₂-C₄) alkylamino, imidazolinium and ammonium radicals.

Among the para-phenylenediamines of formula (II) above, mention may be made more particularly of para-phenylenediamine, para-tolylenediamine, 2-chloro-para-phenylenediamine, 2,3-dimethyl-para-phenylene-diamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-amino-N,N-bis(β-hydroxyethyl)-2-methylaniline, 4-amino-2-chloro-N,N-bis(β-hydroxyethyl)aniline, 2-β-hydroxy-ethyl-para-phenylenediamine, 2-fluoro-para-phenylene-diamine, 2-isopropyl-para-phenylenediamine, N-(β-hydroxypropyl)-para-phenylenediamine, 2-hydroxy-methyl-para-phenylenediamine, N,N-dimethyl-3-methyl-para-phenylenediamine, N,N-(ethyl-p-hydroxyethyl)-para-phenylenediamine, N-(β,γ-dihydroxypropyl)-para-phenylenediame, N-(4′-aminophenyl)-para-phenylene-diamine, N-phenyl-para-phenylenediamine, 2-β-hydroxy-ethyloxy-para-phenylenediamine, 2-β-acetylamino-ethyloxy-para-phenylenediamine, N-(β-methoxyethyl)-para-phenylenediamine, and 2-methyl-1-N-β-hydroxyethyl-para-phenylenediamine and the addition salts thereof with an acid.

Among the para-phenylenediamines of formula (II) above, para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, and 2-chloro-para-phenylenediamine and the addition salts thereof with an acid are most particularly preferred.

According to the invention, the term “double bases” is understood to refer to the compounds containing at least two aromatic rings bearing amino and/or hydroxyl groups.

Among the double bases which can be used as oxidation bases in the dye compositions in accordance with the invention, mention may be made in particular of the compounds corresponding to formula (III) below, and the addition salts thereof with an acid: 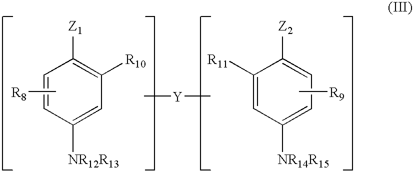
in which:

Z₁ and Z₂, which may be identical or different, represent a hydroxyl or —NH₂ radical which may be substituted with a C₁-C₄ alkyl radical or with a linker arm Y;

the linker arm Y represents a linear or branched alkylene chain containing from 1 to 14 carbon atoms, which may be interrupted or terminated with one or more nitrogenous groups and/or one or more hetero atoms such as oxygen, sulfur or nitrogen atoms, and optionally substituted with one or more hydroxyl or C₁-C₆ alkoxy radicals;

R₈ and R₉ represent a hydrogen or halogen atom, a C₁-C₄ alkyl radical, a C₁-C₄ monohydroxyalkyl radical, a C₂-C₄ polyhydroxyalkyl radical, a C₁-C₄ aminoalkyl radical or a linker arm Y;

R₁₀, R₁₁, R₁₂, R₁₃, R₁₄ and R₁₅, which may be identical or different, represent a hydrogen atom, a linker arm Y or a C₁-C₄ alkyl radical;

it being understood that the compounds of formula (III) contain only one linker arm Y per molecule.

Among the nitrogenous groups of formula (III) above, mention may be made in particular of amino, mono(C₁-C₄)alkylamino, di(C₁-C₄)alkylamino, tri(C₁-C₄)-alkylamino, monohydroxy(C₁-C₄)alkylamino, imidazolinium and ammonium radicals.

Among the double bases of formula (III) above, mention may be made more particularly of N,N′-bis(β-hydroxyethyl)-N,N′-bis(4′-aminophenyl)-1,3-diaminopropanol, N,N′-bis(P-hydroxyethyl)-N,N′-bis(4′-aminophenyl)ethylenediamine, N,N′-bis(4-aminophenyl)-tetramethylenediamine, N,N′-bis(P-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)-ethylenediamine, 1,8-bis(2,5-diaminophenoxy)-3,5-dioxaoctane, and the addition salts thereof with an acid.

Among these double bases of formula (III), N,N′-bis(P-hydroxyethyl)-N,N′-bis(4′-aminophenyl)-1,3-diaminopropanol, 1,8-bis(2,5-diaminophenoxy)-3,5-dioxaoctane, or one of the addition salts thereof with an acid, are particularly preferred.

Among the para-aminophenols which can be used as oxidation bases in the dye compositions in accordance with the invention, mention may be made in particular of the compounds corresponding to formula (IV) below, and the addition salts thereof with an acid: 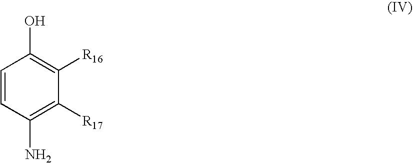
in which:

R₁₆ represents a hydrogen or halogen atom or a C₁-C₄ alkyl, C₁-C₄ monohydroxyalkyl, (C₁-C₄)alkoxy(C₁-C₄)-alkyl, C₁-C₄ aminoalkyl or hydroxy(C₁-C₄)-alkylamino(C₁-C₄)alkyl radical,

R₁₇ represents a hydrogen or halogen atom or a C₁-C₄ alkyl, C₁-C₄ monohydroxyalkyl, C₂-C₄ polyhydroxyalkyl, C₁-C₄ aminoalkyl, C₁-C₄ cyanoalkyl or (C₁-C₄) alkoxy(C₁-C₄) alkyl radical, it being understood that at least one of the radicals R₁₆ or R₁₇ is a hydrogen atom.

Among the para-aminophenols of formula (IV) above, mention may be made more particularly of para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(O-hydroxyethylaminomethyl)phenol, 4-amino-2-fluorophenol, and the addition salts thereof with an acid.

Among the ortho-aminophenols which can be used as oxidation bases in the dye compositions in accordance with the invention, mention may be made more particularly of 2-aminophenol, 2-amino-5-methylphenol, 2-amino-6-methylphenol and 5-acetamido-2-aminophenol, and the addition salts thereof with an acid.

Among the heterocyclic bases which can be used as oxidation bases in the dye compositions in accordance with the invention, mention may be made more particularly of pyridine derivatives, pyrimidine derivatives, pyrazole derivatives, and the addition salts thereof with an acid.

Among the pyridine derivatives, mention may be made more particularly of the compounds described, for example, in patents GB 1 026 978 and GE 1 153 196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine and 3,4-diaminopyridine, and the addition salts thereof with an acid.

Among the pyrimidine derivatives, mention may be made more particularly of the compounds described, for example, in German patent DE 2 359 399 or Japanese patents JP 88-169 571 and JP 91-10659, or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and pyrazolopyrimidine derivatives such as those mentioned in patent application FR-A-2 750 048, and among which mention may be made of pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol; 2-(7-aminopyrazolo[1,5-a]pyrimidin-3-ylamino)ethanol, 2-[(3-aminopyrazolo[1,5-a]pyrimidin-7-yl)-(2-hydroxyethyl)amino]ethanol, 2-[(7-aminopyrazol[1,5-a]pyrimidin-3-yl)-(2-hydroxyethyl)amino]-ethanol, 5,6-dimethylpyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]-pyrimidine-3,7-diamine, 3-amino-5-methyl-7-imidazolylpropylaminopyrazolo[1,5-a]pyrimidine, the tautomeric forms thereof, when a tautomeric equilibrium exists, and the addition salts thereof with an acid.

Among the pyrazole derivatives, mention may be made more particularly of the compounds described in patents DE 3 843 892, DE 4 133 957 and patent applications WO 94/08969, WO 94/08970, FR-A-2 733 749 and DE 195 43 988, such as 4,5-diamino-1-methylpyrazole, 3,4-diaminopyrazole, 4,5-diamino-1-)4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-amino-ethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the addition salts thereof with an acid.

According to one preferred embodiment of the invention, the oxidation base(s) is (are) chosen from para-phenylenediamines.

The oxidation base(s) preferably represent(s) from 0.0005% to 12% by weight approximately relative to the total weight of the dye composition in accordance with the invention, and even more preferably from 0.005% to 8% by weight approximately relative to this weight.

Cationic and Amphoteric Polymers According to the Invention

(i) Among the methacryloyloxyethyltrimethylammonium halide polymers that may be used according to the invention, mention may be made in particular of the products known in the CTFA dictionary (5th edition, 1993) as “Polyquaternium 37”, “Polyquaternium 32” and “Polyquaternium 35”, which correspond, respectively, as regards “Polyquaternium 37”, to crosslinked poly(methacryloyloxyethyltrimethylammonium chloride), as a 50% dispersion in mineral oil, and sold under the name Salcare SC95 by the company Allied Colloids, as regards “Polyquaternium 32”, to the crosslinked copolymer of acrylamide and of methacryloyloxyethyltrimethylammonium chloride (20/80 by weight), as a 50% dispersion in mineral oil, and sold under the name Salcare SC92 by the company Allied Colloids, and as regards “Polyquaternium 35”, to the methosulfate of the copolymer of methacryloyloxyethyltrimethylammonium and of methacryloyloxyethyldimethylacetylammonium, sold under the name Plex 7525L by the company Rohm GmbH.

(ii) The polyquaternary ammoniums of formula (I) that may be used according to the invention may be prepared according to the processes described in U.S. Pat. Nos. 4,157,388, 4,390,689, 4,702,906 and 4,719,282. They are described especially in patent application EP-A-122 324.

Among these polymers, the ones that are preferred are those with a molecular mass measured by carbon-13 NMR of less than 100 000, for which p is equal to 3, and

a) D represents a —(CH₂)₄—CO— group, X denotes a chlorine atom, the molecular mass measured by carbon-13 NMR (¹³C NMR) being about 5600; a polymer of this type is sold by the company Miranol under the name Mirapol-ADI,

b) D represents a —(CH₂)₂—CO— group, X denotes a chlorine atom, the molecular mass measured by carbon-13 NMR (¹³C NMR) being about 8100; a polymer of this type is sold by the company Miranol under the name Mirapol-AZI,

c) D denotes the value zero, X denotes a chlorine atom, the molecular mass measured by carbon-13 NMR (¹³C NMR) being about 25 500; a polymer of this type is sold by the company Miranol under the name Mirapol-A15,

d) a “block copolymer” formed from units corresponding to the polymers described in paragraphs a) and c), sold by the company Miranol under the names Mirapol-9 (¹³C NMR molecular mass, about 7800), Mirapol-175 (¹³C NMR molecular mass, about 8000) and Mirapol-95 (¹³C NMR molecular mass, about 12 500).

Even more particularly, the polymer that is preferred according to the invention is a polymer containing units of formula (I) in which p is equal to 3, D denotes the value zero, X denotes a chlorine atom, the molecular mass measured by carbon-13 NMR (¹³C NMR) being about 25 500.

Among the polyquaternary ammoniums of formula (II) that may be used according to the invention, the ones that are preferred are those of formula (II) in which R₁, R₂, R₃ and R₄ represent a methyl or ethyl radical.

Polymers of formula (II) that are particularly preferred are those for which R₁, R₂, R₃ and R₄ represent a methyl radical and n=3, p=6 and X═Cl, and especially those with a molecular weight, determined by gel permeation chromatography, of between 9500 and 9900 [polymer W].

Other polymers of formula (II) that are particularly preferred are those for which R₁ and R₂ represent a methyl radical, R₃ and R₄ represent an ethyl radical and n=p=3 and X═Br, and especially those with a molecular weight, determined by gel permeation chromatography, of about 1200 [polymer U].

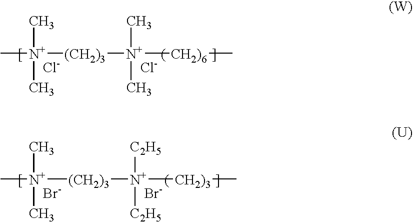

Said polymers containing units (W) and (U) are prepared and described in French patent 2 270 846.

(iii) Among the vinylpyrrolidone polymers (PVP) containing cationic units which can be used in accordance with the invention, mention may be made in particular of:

a) vinylpyrrolidone polymers containing dimethylaminoethyl methacrylate units; among these, mention may be made of:

the vinylpyrrolidone/dimethylaminoethyl methacrylate (20/80 by weight) copolymer sold under the trade name Copolymer 845 by the company ISP,

the vinylpyrrolidone/dimethylaminoethyl methacrylate copolymers quaternized with diethyl sulfate, sold under the names Gafquat 734, 755, 755 S and 755 L by the company ISP,

the PVP/dimethylaminoethyl methacrylate/hydrophilic polyurethane copolymers sold under the trade name Pecogel GC-310 by the company UCIB or alternatively under the names Aquamere C 1031 and C 1511 by the company Blagden Chemicals,

the quaternized or non-quaternized PVP/dimethylaminoethyl methacrylate/C8 to C16 olefin copolymers sold under the names Ganex ACP 1050 to 1057, 1062 to 1069 and 1079 to 1086 by the company ISP,

the PVP/dimethylaminoethyl methacrylate/vinylcaprolactam copolymer sold under the name Gaffix VC 713 by the company ISP.

b) vinylpyrrolidone polymers comprising methacrylamido-propyltrimethylammonium (MAPTAC) units, among which mention may be made in particular of:

the vinylpyrrolidone/MAPTAC copolymers sold under the trade names Gafquat ACP 1011 and Gafquat HS 100 by the company ISP.

c) vinylpyrrolidone polymers containing methylvinylimidazolium units, and among which mention may be made more particularly of:

the PVP/methylvinylimidazolium chloride copolymers sold under the names Luviquat FC 370, FC 550, FC 905 and HM 552 by the company BASF,

the PVP/methylvinylimidazolium chloride/vinylimidazole copolymer sold under the name Luviquat 8155 by the company BASF,

the PVP/methylvinylimidazolium methosulfate copolymer sold under the name Luviquat MS 370 by the company BASF.

(iv) According to the invention, the expression “amino silicone” denotes any silicone comprising at least one primary, secondary or tertiary amine or a quaternary ammonium group.

In accordance with the invention, the amino silicones are chosen from:
(i) compounds known in the CTFA dictionary as “amodimethicone” and corresponding to formula (VIII) below: 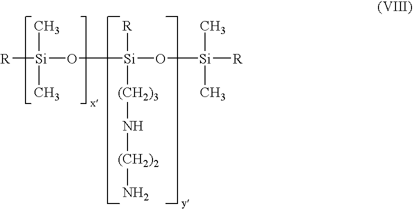
   in which R denotes a CH₃ or OH radical, and
   x′ and y′ are integers depending on the molecular weight, generally such that said number-average molecular weight is between 5000 and 500 000 approximately;
(ii) the compounds corresponding to formula (IX) below:
   (R¹)ₐ(T)₃₋ₐSi[OSi(T)₂]ₙ—[OSi(T)_{b}(R¹)_{3-b}]ₘ—OSi(T)₃₋ₐ—(R¹)ₐ (IX)
   in which
   T is a hydrogen atom or a phenyl or OH or C₁-C₈ alkyl radical, and preferably methyl,
   a denotes the number 0 or an integer from 1 to 3, and preferably 0,
   b denotes 0 or 1, and in particular 1,
   m and n are numbers such that the sum (n+m) can range especially from 1 to 2000 and in particular from 50 to 150, n possibly denoting a number from 0 to 1999 and especially from 49 to 149, and m possibly denoting a number from 1 to 2000 and especially from 1 to 10;
   R¹ is a monovalent radical of formula —C_{q}H_{2q}L in which q is a number from 2 to 8 and L is an optionally quaternized amino group chosen from the following groups:
   —N(R²)—CH₂—CH₂—N(R²)₂
   —N(R²)²
   —N^{⊕}(R²)₃Q⁻
   —N^{⊕}(R²)(H)₂Q
   —N^{⊕}(R²)₂HQ⁻
   —N(R²)—CH₂—CH₂—N^{⊕} (R²) (H)₂Q⁻
   in which R² can denote hydrogen, phenyl, benzyl or a saturated monovalent hydrocarbon radical, for example an alkyl radical containing from 1 to 20 carbon atoms, and Q⁻ represents a halide ion such as, for example, fluoride, chloride, bromide or iodide.

A product corresponding to this definition is the polymer known in the CTFA dictionary as “trimethylsilylamodimethicone”, corresponding to the formula (X) below: 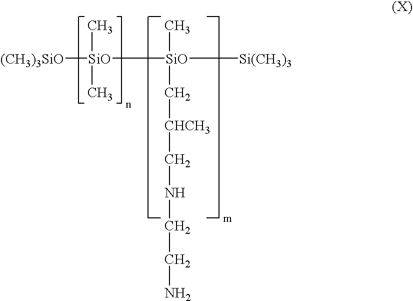
in which n and m have the meanings given above [cf. formula (IX)].

Such compounds are described, for example, in patent application EP-A-95238; a compound of formula (X) is sold, for example, under the name Q2-8220 by the company OSI.
(iii) the compounds corresponding to formula (XI) below: 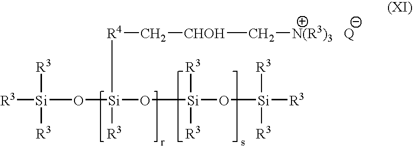
   in which
   R³ represents a monovalent hydrocarbon radical containing from 1 to 18 carbon atoms, and in particular a C₁-C₁₈ alkyl or C₁-C₁₈ alkenyl radical,
   R⁴ represents a divalent hydrocarbon radical, especially a C₁-C₁₈, alkylene radical or a divalent C₁-C₁₈, and for example C₁-C₈, alkylenoxy radical;
   Q⁻ is a halide ion, especially chloride;
   r represents an average statistical value from 2 to 20 and in particular from 2 to 8;
   s represents an average statistical value from 20 to 200 and in particular from 20 to 50.

Such compounds are described more particularly in patent U.S. Pat. No. 4,185,087. A compound falling within this class is the product sold by the company Union Carbide under the name “Ucar Silicone ALE 56”.

When these amino silicones are used, one particularly advantageous embodiment is their joint use with cationic and/or nonionic surfactants.

Use may be made, for example, of the product sold under the name “Cationic Emulsion DC 939” by the company Dow Corning, which comprises, besides amodimethicone, a cationic surfactant which is trimethylcetylammonium chloride and a nonionic surfactant of formula:
C₁₃H₂₇—(OC₂H₄)₁₂—OH,
known under the CTFA name “trideceth-12”.

Another commercial product that may be used according to the invention is the product sold under the name “Dow Corning Q2 7224” by the company Dow Corning, comprising, in combination, the trimethylsilylamodimethicone of formula (X) described above, a nonionic surfactant of formula:
C₈H₁₇—C₆H₄—(OCH₂CH₂)₄₀13 OH,
known under the CTFA name “octoxynol-40”, a second nonionic surfactant of formula:
C₁₂H₂₅—(OCH₂—CH₂)₆—OH,
known under the CTFA name “isolaureth-6”, and propylene glycol.
(iv) the compounds of formula (XII) below: 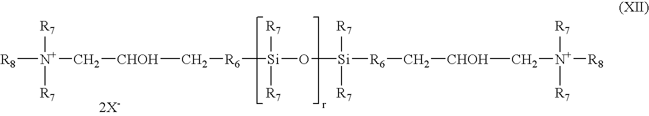
   in which:
   R₆ represents a divalent hydrocarbon radical, especially a C₃-C₁₈ alkylene radical or a divalent C₁-C₁₈ alkylenoxy radical linked to the Si via an SiC bond; the radicals R₇, which may be identical or different, represent a monovalent hydrocarbon radical containing from 1 to 18 carbon atoms, a C₂-C₁₈ alkenyl radical or a ring containing 5 or 6 carbon atoms; the radicals R₈, which may be identical or different, represent a hydrogen atom, a monovalent hydrocarbon radical containing from 1 to 18 carbon atoms, a C₂-C₁₈ alkenyl radical or a radical —R₆—NHCOR₇;
   r represents an average statistical value from 2 to 200;
   X⁻ is an anion such as a halide ion, especially chloride, or an organic acid salt (acetate, etc.).

The cationic silicones of formula (XII) are described, for example, in patent application EP-A-0 530 974.

Silicones forming part of this class are the silicones sold by the company Goldschmidt under the names Abil Quat 3270, Abil Quat 3272 and Abil Quat 3474.
(v) Among the dimethyldiallylammonium halide homopolymers that may be mentioned in particular are those of dimethyldiallylammonium chloride, such as-the product sold under the name “Merquat 100” by the company Merck.

Among the copolymers of dimethyldiallylammonium halide and of acrylamide, mention may be made in particular of those of dimethyldiallylammonium chloride and of acrylamide, sold under the names “Merquat 550” and “Merquat S” by the company Merck.
(vi) Among the copolymers of dimethyldiallylammonium halide and of acrylic acid, mention may be made in particular of those of dimethyldiallylammonium chloride and of acrylic acid, for instance those sold under the names “Merquat 280”, “Merquat 295” and “Merquat Plus 3330” by the company Calgon.
(vii) and (viii) The copolymers of (meth)acrylic acid or of maleic acid or of α-chloroacrylic acid and of dialkylaminoalkyl (meth)acrylate or of dialkylaminoalkyl(meth)acrylamide are described in patent U.S. Pat. No. 3,836,537.

The sodium acrylate/acrylamidopropyl-trimethylammonium chloride copolymer is sold under the name “Polyquart KE 3033” by the company Henkel.
(ix) Octylacrylamide/acrylates/butylaminoethyl methacrylate copolymer is a sold under the name “Amphomer” or “Lovocryl 47” by the company CTFA name (4th ed., 1991) and covers products such as those National Starch.
(x) The polymers comprising zwitterionic units of formula (III) may also comprise units derived from nonzwitterionic monomers such as dimethylaminoethyl or diethylaminoethyl acrylate or methacrylate or alkyl acrylates or methacrylates, acrylamides or methacrylamides or vinyl acetate.

By way of example, mention may be made of the copolymer of methyl methacrylate/methyl dimethylcarboxymethylammonioethyl methacrylate, such as the product sold under the name “Diaformer Z301” by the company Sandoz.
(xiii) The (C₁-C₅)alkyl vinyl ether/maleic anhydride copolymers are partially modified by semiamidation with an N,N-dialkylaminoalkylamine such as N,N-dimethylaminopropylamine, or by semiesterification with an N,N-dialkanolamine. They may also comprise other vinyl comonomers such as vinylcaprolactam.

The cationic or amphoteric polymer(s) thus defined, which may be used in the dye composition of the present invention, may represent from 0.01% to 10% by weight approximately, preferably from 0.05% to 5% by weight approximately and in particular from 0.1% to 3% by weight approximately relative to the total weight of the composition.

The dye composition according to the invention may also contain one or more additional couplers other than the 3,5-diaminopyridines of formula (I) according to the invention and chosen from meta-phenylenediamines, meta-aminophenols, meta-diphenols, naphthols and heterocyclic couplers such as pyrazolo[1,5-b]-1,2,4-triazoles, pyrazolo[3,2-c]-1,2,4-triazoles, pyrazol-5-ones, pyridines other than the 3,5-diaminopyridines of formula (I) according to the invention, indoles, indolines, indazoles, benzimidazoles, benzothiazoles, benzoxazoles, 1,3-benzodioxoles and quinolines.

These couplers are more particularly chosen from 2,4-diamino-1-(β-hydroxyethyloxy)benzene, 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2-amino-4-(β-hydroxyethylamino)-1-methoxybenzene, 1,3-diaminobenzene, 1,3-bis(2,4-diaminophenoxy)propane, sesamol, α-naphthol, 2-methyl-1-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, 2-amino-3-hydroxypyridine, 3,6-dimethylpyrazolo[3,2-c]-1,2,4-triazole, 2,6-dimethylpyrazolo[1,5-b]-1,2,4-triazole and 3-(4-hydroxy-1-methyl-1H-indol-5-ylmethyl)-1-methylpyridinium, and the addition salts thereof with an acid.

When they are present, the additional coupler(s) preferably represent(s) from 0.0001% to 10% by weight approximately relative to the total weight of the dye composition in accordance with the invention, and even more preferably from 0.005% to 5% by weight approximately relative to this weight.

The addition salts with an acid of the 3,5-diaminopyridines of formula (I), of the oxidation dye precursors and of the additional couplers that may be present, and which may be used in the dye compositions according to the invention, are chosen especially from the hydrochlorides, hydrobromides, sulfates, tartrates, lactates and acetates.

The dye composition according to the invention may also contain direct dyes used especially to modify the shades by enriching them with glints; in this case, they may be chosen from neutral, cationic or anionic nitro dyes, azo dyes or anthraquinone dyes in a weight proportion of from 0.001% to 20% approximately and preferably from 0.01% to 10% relative to the total weight of the composition.

The medium for the composition that is suitable for dyeing is preferably an aqueous medium consisting of water and can advantageously contain cosmetically acceptable organic solvents, including, more particularly, alcohols such as ethyl alcohol, isopropyl alcohol, benzyl alcohol and phenylethyl alcohol, or glycols or glycol ethers such as, for example, ethylene glycol monomethyl, monoethyl or monobutyl ether, propylene glycol or its ethers, such as, for example, propylene glycol monomethyl ether, butylene glycol, dipropylene glycol, and also diethylene glycol alkyl ethers such as, for example, diethylene glycol monoethyl ether or monobutyl ether, in concentrations ranging from about 0.5% to about 20% and preferably between about 2% and 10% by weight relative to the total weight of the composition.

The composition according to the invention may also contain an effective amount of other agents, known previously elsewhere in oxidation dyeing, such as various common adjuvants, for instance sequestering agents such as EDTA and etidronic acid, UV screening agents, waxes, volatile or non-volatile, cyclic or linear or branched silicones, preserving agents, ceramides, pseudoceramides, plant, mineral or synthetic oils, vitamins or provitamins, for instance panthenol, opacifiers, etc.

Said composition may also contain reducing agents or antioxidants. These agents may be chosen in particular from sodium sulfite, thioglycolic acid, thiolactic acid, sodium bisulfite, dehydroascorbic acid, hydroquinone, 2-methylhydroquinone, tert-butylhydroquinone, 1-phenyl-3-methylpyrazolone and homogentisic acid, and, in this case, they are generally present in amounts ranging from about 0.05% to 1.5% by weight relative to the total weight of the composition.

The composition according to the invention can also contain one or more fatty alcohols, these fatty alcohols being introduced in pure form or as a mixture. Among these, mention may be made more particularly of lauryl alcohol, cetyl alcohol, stearyl alcohol and oleyl alcohol and mixtures thereof. These fatty alcohols can represent from 0.001% to 20% by weight approximately relative to the total weight of the composition.

Preferably, the dye composition and/or the oxidizing composition of the ready-to-use composition according to the invention contains at least one nonionic, anionic, cationic or amphoteric surfactant in a proportion of from 0.1% to 20% by weight approximately.

Even more preferably, said composition contains at least one nonionic surfactant.

Needless to say, a person skilled in the art will take care to select the optional additional compound(s) mentioned above such that the advantageous properties intrinsically associated with the oxidation dye composition according to the invention are not, or are not substantially, adversely affected by the envisaged addition(s).

In the oxidizing composition, the oxidizing agent is preferably chosen from hydrogen peroxide, urea peroxide, alkali metal bromates or ferricyanides, and persalts such as perborates and persulfates. It is particularly preferred to use hydrogen peroxide. This oxidizing agent advantageously consists of an aqueous hydrogen peroxide solution whose titre may range, more particularly, from about 1 to 40 volumes and even more preferably from about 5 to 40 volumes.

Oxidizing agents which may also be used are one or more redox enzymes such as laccases, peroxidases and 2-electron oxidoreductases, where appropriate in the presence of their respective donor or co-factor.

The pH of the dye composition or of the ready-to-use composition applied to the keratin fibers [composition resulting from mixing together the dye composition according to the invention and the oxidizing composition] is generally between 3 and 12. It is preferably between 6 and 11 and may be adjusted to the desired value using acidifying or basifying agents that are well known in the prior art in the dyeing of keratin fibers.

Among the basifying agents which may be mentioned, for example, are aqueous ammonia, alkali metal carbonates, alkanolamines such as monoethanolamine, diethanolamine and triethanolamine and derivatives thereof, oxyethylenated and/or oxypropylenated hydroxyalkylamines and ethylenediamines, sodium hydroxide, potassium hydroxide and the compounds of formula (XIII) below: 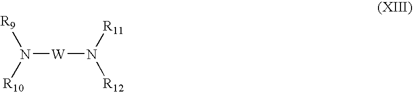
in which W is a propylene residue optionally substituted with a hydroxyl group or a C₁-C₄ alkyl radical; R₉, R₁₀, R₁₁ and R₁₂, which may be identical or different, represent a hydrogen atom, a C₁-C₄ alkyl radical or a C₁-C₄ hydroxyalkyl radical.

The acidifying agents are conventionally, for example, mineral or organic acids, for instance hydrochloric acid, orthophosphoric acid, carboxylic acids, for instance tartaric acid, citric acid or lactic acid, or sulfonic acids.

The dye composition in accordance with the invention may be in various forms, such as in the form of liquids, powders, creams or gels, that may be pressurized, or in any other form that is suitable for dyeing keratin fibers, and especially human hair.

The dyeing process according to the invention preferably consists in applying the ready-to-use composition, prepared extemporaneously at the time of use from the dye composition according to the invention and the oxidizing composition described above, to wet or dry keratin fibers, and in leaving the composition to act for an exposure time preferably ranging from 1 to 60 minutes approximately, and more preferably from 10 to 45 minutes approximately, in rinsing the fibers and then in optionally washing them with shampoo, rinsing them again and then drying them.

The example that follows is intended to illustrate the invention.

### EXAMPLE

The dye composition below was prepared: (expressed in grams—AM* denotes Active Material)

| | |
|---|---|
| 2,6-Dimethoxy-3,5-diaminopyridine dihydrochloride | 0.363 |
| (coupler of formula (I)) | |
| para-Phenylenediamine (oxidation base) | 0.324 |
| 2,4-Diamino-1-(β-hydroxyethyloxy)benzene | 0.361 |
| dihydrochloride (additional coupler) | |
| Polyquaternary ammonium of formula (W) according | 2.16 AM* |
| to the invention | |
| Dye support | (*) |

| (*) Dye support | |
|---|---|
| Octyldodecanol sold under the name Eutanol D by | 8 |
| the company Henkel | |
| Oleic acid | 20 |
| Monoethanolamine lauryl ether sulfate sold under | 3 |
| the name Sipon LM 35 by the company Henkel | |
| Ethyl alcohol | 10 |
| Benzyl alcohol | 10 |
| Cetylstearyl alcohol containing 33 mol of ethylene | 2.4 |
| oxide, sold under the name Simulsol GS by the | |
| company SEPPIC | |
| Ethylenediaminetetraacetic acid | 0.2 |
| Monoethanolamine | 7.5 |
| Linoleic acid diethanolamide sold under the name | 8 |
| Comperlan F by the company Henkel | |
| Aqueous ammonia containing 20% NH₃ | 10.2 |
| Sodium metabisulfite as an aqueous 35% solution | 0.45 AM* |
| Hydroquinone | 0.15 |
| 1-Phenyl-3-methyl-5-pyrazolone | 0.2 |
| Demineralized water | qs 100 |

The dye composition described above was mixed weight-for-weight, at the time of use, with a 20-volumes hydrogen peroxide solution (6% by weight) of pH 3.

The mixture thus prepared was applied for 30 minutes to a lock of permanent-waved natural gray hair containing 90% white hairs. The lock was then rinsed, washed with a standard shampoo, rinsed again and then dried. It was dyed in a very ash dark chestnut shade.

1-32. (canceled)

33. A cosmetic composition for the oxidation dyeing of keratin fibers comprising, in a cosmetically acceptable medium:
(a) at least one coupler chosen from compounds of formula (I) and acid addition salts thereof: 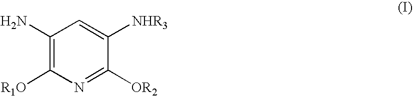
wherein:
R₁ and R₂, which may be identical or different, are chosen from C₁-C₄ alkyl, C₁-C₄ monohydroxyalkyl, and C₂-C₄ polyhydroxyalkyl radicals,
R₃ is chosen from hydrogen and C₁-C₄ alkyl, C₁-C₄ monohydroxyalkyl, and C₂-C₄ polyhydroxyalkyl radicals;
and
(b) at least one oxidation dye precursor,
wherein the composition further comprises at least one polymer chosen from cationic and amphoteric polymers chosen from:
(i) methacryloyloxyethyltrimethylammonium halide homopolymers and copolymers;
(ii) polyquaternary ammoniums comprising repeating units of formula (I′) or (II): 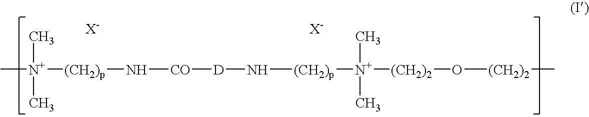
wherein p is an integer ranging from 1 to 6, D is chosen from a direct bond and a group —(CH₂)ᵣ—CO— wherein r is a number equal to 4 or to 7, and X⁻ is an anion; 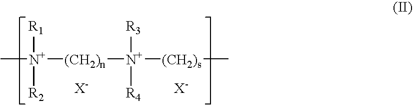
wherein R₁, R₂, R₃ and R₄, which may be identical or different, are chosen from alkyl and hydroxyalkyl radicals containing 1 to 4 carbon atoms, n and s are integers ranging from 2 to 20, and X⁻ is an anion;
(iii) vinylpyrrolidone copolymers comprising cationic units;
(iv) amino silicones;
(v) dimethyldiallylammonium halide homopolymers and copolymers of dimethyldiallylammonium halide and of acrylamide;
(vi) copolymers of dimethyldiallylammonium halide and of acrylic acid;
(vii) copolymers of (meth)acrylic acid, or of maleic acid, or of α-chloroacrylic acid and of dialkylaminoalkyl (meth)acrylate or of dialkylaminoalkyl(meth)acrylamide;
(viii) the copolymer of sodium acrylate and of acrylamidopropyltrimethylammonium chloride;
(ix) octylacrylamide/acrylates/butylaminoethyl methacrylate copolymer;
(x) polymers comprising at least one zwitterionic unit of formula (III): 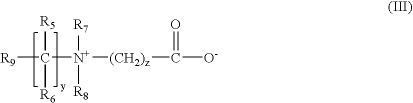
wherein R₉ is chosen from acrylate, methacrylate, acrylamide, and methacrylamide groups,
y and z, which may be identical or different, are chosen from an integer ranging from 1 to 3,
R₅ and R₆, which may be identical or different, are chosen from hydrogen and methyl, ethyl and propyl groups,
R₇ and R₈, which may be identical or different, are chosen from hydrogen and alkyl radicals such that the sum of the carbon atoms in R₇ and R₈ does not exceed 10;
(xi) polymers derived from chitosan comprising monomer units corresponding to formulae (IV), (V) and (VI) below combined in their chain: 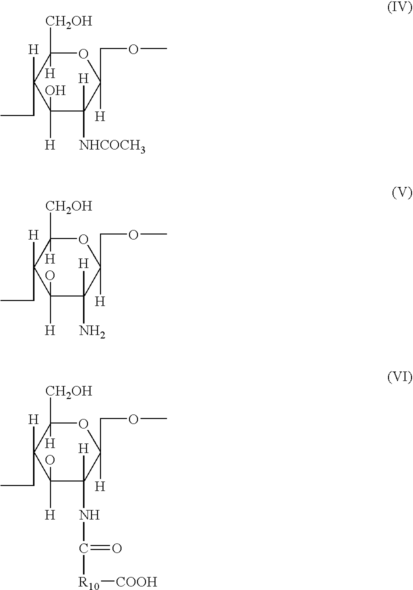
the unit (IV) being present in an amount ranging from 0 to 30%, the unit (V) being present in an amount ranging from 5% to 50%, and the unit (VI) being present in an amount ranging from 30% to 90%, wherein in the unit (VI), R₁₀ is chosen from radicals of formula: 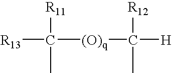
wherein q is zero or 1;
if q=0, R₁₁, R₁₂ and R₁₃, which may be identical or different, are each chosen from hydrogen, methyl, hydroxyl, acetoxy and amino residues, monoalkylamine residues, and dialkylamine residues optionally interrupted by at least one nitrogen atom and optionally substituted with at least one group chosen from amine, hydroxyl, carboxyl, alkylthio and sulfonic groups, alkylthio residues wherein the alkyl group bears an amino residue, at least one of the radicals R₁₁, R₁₂ and R₁₃ being in this case a hydrogen atom;
and if q=1, R₁₁, R₁₂ and R₁₃ are each a hydrogen atom,
and the salts formed by these compounds with bases or acids;
(xii) polymers corresponding to formula (VII): 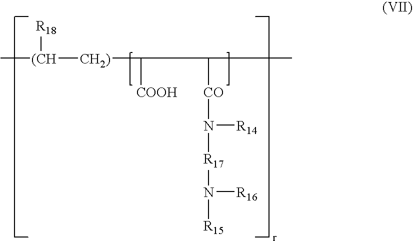
wherein R₁₈ is chosen from hydrogen, CH₃O, CH₃CH₂O and phenyl radicals,
R₁₄ is chosen from hydrogen and (C₁-C₆)alkyl radicals,
R₁₅ is chosen from hydrogen and (C₁-C₆)alkyl radicals,
R₁₆ is chosen from (C₁-C₆)alkyl radicals, and radicals corresponding to the formula: —R₁₇—N(R₁₅)₂, wherein R₁₇ is chosen from —CH₂—CH₂—, —CH₂—CH₂—CH₂— and —CH₂—CH(CH₃)— groups, and wherein R₁₅ is chosen from hydrogen and C₁-C₆ alkyl radicals, and
r is an integer greater than 1; and
(xiii) copolymers of (C₁-C₅)alkyl vinyl ether/maleic anhydride partially modified by semiamidation with an N,N-dialkylaminoalkylamine or by semiesterification with an N,N-dialkanolamine.

34. The composition according to claim 33, wherein said keratin fibers are human hair.

35. The composition according to claim 33, wherein the at least one coupler is chosen from 2,6-dimethoxy-3,5-diaminopyridine, 2,6-diethoxy-3,5-diaminopyridine, 2,6-di(P-hydroxyethyloxy)-3,5-diaminopyridine, and acid addition salts thereof.

36. The composition according to claim 35, wherein the at least one coupler is chosen from 2,6-dimethoxy-3,5-diaminopyridine and acid addition salts thereof.

37. The composition according to claim 33, wherein the at least one coupler is present in an amount ranging from 0.0001% to 10% by weight, relative to the total weight of the composition.

38. The composition according to claim 37, wherein the at least one coupler is present in an amount ranging from 0.005% to 5% by weight, relative to the total weight of the composition.

39. The composition according to claim 33, wherein the at least one oxidation dye precursor is chosen from para-phenylenediamines, double bases, para-aminophenols, ortho-aminophenols, heterocyclic oxidation bases, and acid addition salts thereof.

40. The composition according to claim 33, wherein the at least one oxidation dye precursor is present in an amount ranging from 0.0005% to 12% by weight, relative to the total weight of the composition.

41. The composition according to claim 33, wherein the polyquaternary ammoniums are chosen from those of formula (I′) for which p is equal to 3, X is chlorine and D is chosen from a direct bond, a —(CH₂)₄—CO— group, and a —(CH₂)₇—CO— group.

42. The composition according to claim 33, wherein the polyquaternary ammoniums are chosen from those of formula (II) for which:
R₁, R₂, R₃ and R₄ are methyl radicals and n=3, s=6 and X═Cl [polymer W];
or R₁ and R₂ are methyl radicals, R₃ and R₄ are ethyl radicals and n=s=3 and X═Br, [polymer U]:

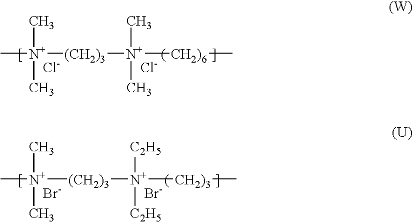

43. The composition according to claim 33, wherein the vinylpyrrolidone polymers containing cationic units are chosen from vinylpyrrolidone polymers containing dimethylaminoethyl methacrylate units, vinylpyrrolidone/dimethylaminoethyl methacrylate copolymers quaternized with diethyl sulfate, polyvinylpyrrolidone/dimethylaminoethyl methacrylate/hydrophilic polyurethane copolymers, quaternized or nonquaternized polyvinylpyrrolidone/dimethylaminoethyl methacrylate/C8 to C16 olefin copolymers, polyvinylpyrrolidone/dimethylaminoethyl methacrylate/vinylcaprolactam copolymers, vinylpyrrolidone polymers comprising methacrylamidopropyltrimethylammonium (MAPTAC) units, and vinylpyrrolidone polymers comprising methylvinylimidazolium units.

44. The composition according to claim 33, wherein the amino silicones are chosen from:
(i) a amodimethicone of formula (VIII): 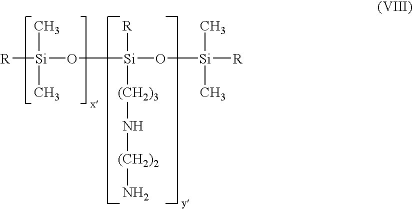
wherein R is chosen from CH₃ and OH radicals, and
x′ and y′ are integers depending on the molecular weight, such that said number-average molecular weight ranges from 5000 to 500 000;
(ii) the compounds of formula (IX):
(R¹)ₐ(T)₃₋ₐSi[OSi(T)₂]ₙ—[OSi(T)_{b})R¹)_{2-b}]ₘ—OSi(T)₃₋ₐ—(R¹)ₐ (IX)
wherein
T is chosen from hydrogen and phenyl, OH, and C₁-C₈ alkyl radicals,
a is chosen from 0 and integers ranging from 1 to 3,
b is chosen from 0 and 1,
m and n are numbers such that the sum (n+m) ranges from 1 to 2000, n ranges from 0 to 1999, and m ranges from 1 to 2000;
R¹ is a monovalent radical of formula —C_{q}H_{2q}L wherein q is a number ranging from 2 to 8 and L is an optionally quaternized amino group chosen from:
—N(R²)—CH₂—CH₂—N(R²)₂
—N(R²)₂
—N^{⊕}(R²)₃Q⁻
—N^{⊕}(R²)(H)₂Q⁻
—N^{⊕}(R²)₂HQ⁻
—N(R²)—CH₂—CH₂—N^{⊕}(R²)(H)₂Q⁻
wherein R² is chosen from hydrogen, phenyl, benzyl and alkyl radicals containing from 1 to 20 carbon atoms, and Q0 is a halide ion;
(iii) the compounds of formula (XI): 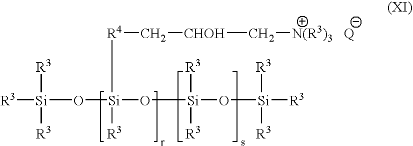
wherein
R³ is chosen from C₁-C₁₈ alkyl and C₂-C₁₈ alkenyl radicals,
R⁴ is chosen from a C₁-C₁₈ alkylene and divalent C₁-C₁₈ alkylenoxy radicals,
Q⁻ is a halide ion,
r is an average statistical value ranging from 2 to 20,
s is an average statistical value ranging from 20 to 200;
(iv) the compounds of formula (XII): 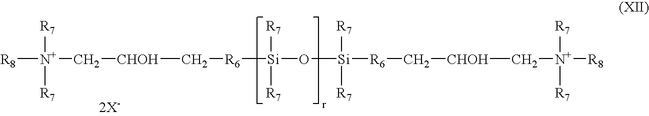
wherein:
R₆ is chosen from C₁-C₁₈ alkylene and divalent C₁-C₁₈ alkylenoxy radicals linked to the Si via an SiC bond,
the radicals R₇, which may be identical or different, are chosen from monovalent hydrocarbon radicals containing from 1 to 18 carbon atoms, C₂-C₁₈ alkenyl radicals and rings containing 5 or 6 carbon atoms,
the radicals R₈, which may be identical or different, are chosen from hydrogen, monovalent hydrocarbon radicals containing from 1 to 18 carbon atoms, C₂-C₁₈ alkenyl radicals and —R₆—NHCOR₇ radicals,
r is an average statistical value ranging from 2 to 200,
X⁻ is an anion.

45. The composition according to claim 44, wherein T of formula (IX) is a methyl radical.

46. The composition according to claim 44, wherein the amino silicone of formula (IX) is trimethylsilylamodimethicone.

47. The composition according to claim 44, wherein the amodimethicone is combined with trimethylcetylammonium-chloride and with Trideceth-12.

48. The composition according to claim 46, wherein the trimethylsilylamodimethicone is combined with Octoxynol-40 and Isolaureth-6.

49. The composition according to claim 33, wherein the at least one polymer chosen from cationic and amphoteric polymers is present in an amount ranging from 0.01% to 10% by weight, relative to the total weight of the composition.

50. The composition according to claim 49, wherein the at least one polymer chosen from cationic and amphoteric polymers is present in an amount ranging from 0.05% to 5% by weight, relative to the total weight of the composition.

51. The composition according to claim 50, wherein the at least one polymer chosen from cationic and amphoteric polymers is present in an amount ranging from 0.1% to 3% by weight, relative to the total weight of the composition.

52. The composition according to claim 33, further comprising at least one additional coupler.

53. The composition according to claim 52, wherein the at least one additional coupler is chosen from meta-phenylenediamines, meta-aminophenols, meta-diphenols, naphthols, heterocyclic couplers other than the compounds of formula (I), and the acid addition salts of these compounds.

54. The composition according to claim 52, wherein the at least one additional coupler is present in an amount ranging from 0.0001% to 10% by weight, relative to the total weight of the composition.

55. The composition according to claim 33, wherein the acid addition salts of the compounds of formula (I) and of the oxidation dye precursors are independently chosen from hydrochlorides, hydrobromides, sulfates, tartrates, lactates and acetates.

56. The composition according to claim 52, wherein the acid addition salts of the at least one additional coupler are chosen from hydrochlorides, hydrobromides, sulfates, tartrates, lactates and acetates.

57. The composition according to claim 33, further comprising at least one direct dye.

58. The composition according to claim 57, wherein the at least one direct dye is present in an amount ranging from 0.001% to 20% by weight, relative to the total weight of the composition.

59. The composition according to claim 58, wherein the at least one direct dye is present in an amount ranging from 0.05% to 1.5% by weight, relative to the total weight of the composition.

60. The composition according to claim 33, further comprising at least one reducing agent in an amount ranging from 0.05% to 1.5% by weight, relative to the total weight of the composition.

61. The composition according to claim 33, further comprising at least one antioxidant in an amount ranging from 0.05% to 1.5% by weight, relative to the total weight of the composition.

62. A ready-to-use composition for the oxidation dyeing of keratin fibers, obtained by mixing together a cosmetic composition for the oxidation dyeing of keratin fibers according to claim 33 and an oxidizing composition comprising at least one oxidizing agent.

63. The ready-to-use composition according to claim 62, wherein said keratin fibers are human hair.

64. The ready-to-use composition according to claim 62, wherein the at least one oxidizing agent is chosen from hydrogen peroxide, urea peroxide, alkali metal bromates, ferricyanides, persalts, redox enzymes, peroxidases and 2-electron oxidoreductases, where appropriate in the presence of their respective donor or cofactor.

65. The ready-to-use composition according to claim 64, wherein the redox enzymes are laccases.

66. The ready-to-use composition according to claim 62, wherein the at least one oxidizing agent is hydrogen peroxide.

67. The ready-to-use composition according to claim 62, wherein the at least one oxidizing agent is an aqueous hydrogen peroxide solution with a titer ranging from 1 to 40 volumes.

68. The ready-to-use composition according to claim 62, wherein the composition has a pH ranging from 3 to 12.

69. The ready-to-use composition according to claim 62, wherein the cosmetic composition for oxidation dyeing of keratin fibers and/or the oxidizing composition further comprises at least one surfactant chosen from anionic, cationic, nonionic and amphoteric surfactants in an amount ranging from 0.1% to 20% by weight, relative to the total weight of the composition.

70. A process for the oxidation dyeing of keratin fibers, comprising applying to the fibers a dye composition comprising, in a medium suitable for dyeing:
(A) at least one coupler chosen from compounds of formula (I) according to claim 33, the color being developed at alkaline, neutral or acidic pH with the aid of an oxidizing composition that is mixed with the dye composition just at the time of use, or that is applied sequentially without intermediate rinsing, and
(B) at least one polymer chosen from cationic and amphoteric polymers according to claim 33 , wherein the at least one polymer is present in at least one of the dye composition and the oxidizing composition.

71. The process according to claim 70, wherein said keratin fibers are human hair.

72. A multi-compartment device for the oxidation dyeing of keratin fibers, comprising a first compartment containing at least one coupler chosen from compounds of formula (I) according to claim 33, and a second compartment containing at least one oxidizing agent,
wherein said multi-compartment device further comprises at least one polymer chosen from cationic and amphoteric polymers as defined in claim 33, and wherein said at least one polymer is present in at least one compartment chosen from the first compartment and the second compartment.

73. The device according to claim 72, wherein said keratin fibers are human hair.

74. A multi-compartment device for the oxidation dyeing of keratin fibers, comprising a first compartment comprising at least one coupler chosen from compounds of formula (I) according to claim 33, and a second compartment comprising at least one polymer chosen from cationic and amphoteric polymers according to claim 33, and at least one additional compartment comprising at least one oxidizing agent.

75. The device according to claim 74, wherein said keratin fibers are human hair.
